(19) United States
(12) Patent Application Publication
(10) Pub. No.: US 2006/0034787 A1
(43) Pub. Date: Feb. 16, 2006

(54) Flake-form pigments based on aluminium

(76) Inventor: Patrice Bujard, Courtepin (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/537,724

(22) PCT Filed: Dec. 1, 2003
(86) PCT No.: PCT/EP03/50914

§ 371 Date: Jun. 7, 2005

(30) Foreign Application Priority Data
Dec. 10, 2002 (CH) 2002 2089/02

(51) Int. Cl. A61K 8/26; C09C 1/62

(52) U.S. Cl. 424/63; 106/404; 106/31.9

(57) ABSTRACT

The present invention relates to aluminum flakes, comprising (A1) a layer consisting of SiO_{z}, (B) a layer consisting of aluminum on the layer (A1) and (A2) a layer consisting of SiO_{z} on the layer (B), wherein 0.70≦z≦2.0, to a process for the manufacture thereof and to the use thereof in paints, electrostatic coatings, in ink-jet printing, cosmetics, coatings, printing inks, plastics materials, in glazes for ceramics and glass, in security printing and in the production of interference pigments.

The present invention relates to aluminum flakes comprising

(A1) a layer consisting of SiO_{z},

(B) a layer consisting of aluminum on the layer (A1) and

(A2) a layer consisting of SiO_{z} on the layer (B), wherein 0.70≦z≦2.0, to a process for the manufacture thereof and to the use thereof in paints, electrostatic coatings, in Ink-jet printing, cosmetics, coatings, printing inks, plastics materials, in glazes for ceramics and glass, in security printing and in the production of interference pigments.

Aluminum flakes on which SiO or SiO₂ protective layers are vapor-deposited by means of PVD (physical vapor deposition) are known.

WO00/69975 discloses aluminum flakes comprising

(a) a layer of a dielectric material, such as silicon monoxide or silicon dioxide,

(b) a layer of a metal, such as aluminum,

(c) a layer of a dielectric material, such as silicon monoxide or silicon dioxide. The thickness of the layers of the dielectric is such that the optical properties of the metal are not significantly affected, that is to say is in the range of from 10 to 20 nm.

U.S. Pat. No. 6,013,370 discloses aluminum flakes comprising

(a) a layer of a dielectric material, such as silicon dioxide,

(b) a layer of a metal, such as aluminum,

(c) a layer of a dielectric material, such as silicon dioxide. The thickness of the layers of the dielectric is such that the optical properties of the metal are not significantly affected, that is to say is in the range of from 50 to 200 nm.

In Example 2 of WO00/24946, the manufacture of SiO-coated aluminum flakes is described. According to the description, the thickness of the SiO protective layer is 15 nm or less.

Surprisingly, it has now been found that metal flakes having a brighter appearance and a greater brilliance in comparison with the aluminum flakes known from the prior art can be obtained if the layer thickness of the SiO_{z} layer is in the range of from 200 to 350 nm, preferably from 250 to 300 nm, wherein 0.70≦z≦2.0, preferably 1.4≦z≦2.0.

The present invention accordingly relates to aluminum flakes comprising

(A1) a layer consisting of SiO_{z},

(B) a layer consisting of aluminum on the layer (A1) and

(A2) a layer consisting of SiO_{z} on the layer (B), wherein 0.70≦z≦2.0, preferably 1.4≦z≦2.0, to a process for the manufacture thereof and to the use thereof in paints, electrostatic coatings, in ink-jet printing, cosmetics, coatings, printing inks, plastics materials, in glazes for ceramics and glass, in security printing and In the production of effect pigments.

In a preferred embodiment, the present invention relates to aluminum flakes comprising

(D1) a layer consisting of SiO₂,

(B) a layer consisting of aluminum on the layer (D1) and

(D2) a layer consisting of SiO₂ on the layer (B).

The layer thickness of the SiO_{z} or SiO₂ layer is from 200 to 350 nm, preferably from 250 to 300 nm.

The layer thickness of the layer (B) consisting of aluminum is generally from 10 to 100 nm, preferably from 30 to 50 nm.

The term “SiO_{z} with 0.70≦z≦2.0” means that the molar ratio of oxygen to silicon at the average value of the silicon oxide layer is from 0.70 to 2.0. The composition of the silicon oxide layer can be determined by ESCA (electron spectroscopy for chemical analysis).

The term “SiO_{y} with 0.70≦y≦1.95” means that the molar ratio of oxygen to silicon at the average value of the silicon oxide layer is from 0.70 to 1.95. The composition of the silicon oxide layer can be determined by ESCA (electron spectroscopy for chemical analysis).

According to the present invention the term “aluminum” comprises aluminum and alloys of aluminum. Alloys of aluminum are, for example, described in G. Wassermann in Ullmanns Enzyklopädie der Industriellen Chemie, 4. Auflage, Verlag Chemie, Weinheim, Band 7, S. 281 to 0.292. Especially suitable are the corrosion stable aluminum alloys described on page 10 to 12 of WO00/12634, which comprise besides aluminum silicon, magnesium, manganese, copper, zinc, nickel, vanadium, lead, antimony, tin, cadmium, bismuth, titanium, chromium and/or iron in amounts of less than 20% by weight, preferably less than 10% by weight.

The aluminum flakes have an aluminum core with two substantially parallel faces, the distance between which faces is the shortest axis of the core, the parallel faces, but not the side faces, being coated with silicon oxide. Furthermore, the aluminum flakes coated with silicon oxide have a length of from 2 μm to 5 mm, a width of from 2 μm to 2 mm and a thickness of from 410 to 800 nm and a length to thickness ratio of at least 2:1. The aluminum flakes preferably have lengths and widths of from 1 to 60 μm, preferably from 2 to 40 μm, most preferably from 5 to 20 μm. The length to thickness ratio is from about 2:1 to about 150:1. The length to width ratio is from 3:1 to 1:1.

The silicon oxide/aluminum flakes are not of a uniform shape. Nevertheless, for purposes of brevity, the flakes will be referred to as having a “diameter.” The silicon oxide/aluminum flakes have a high plane-parallelism and a defined thickness in the range of ±10%, especially ±5%, of the average thickness. The silicon oxide/aluminum flakes have a thickness of from 410 to 800 nm, very especially from 530 to 650 nm. It is presently preferred that the diameter of the flakes be in a preferred range of about 2-40 μm with a more preferred range of about 5-20 μm. Thus, the aspect ratio of the flakes of the present invention is in a preferred range of about 8 to 40.

For increasing the stability to weathering and light fastness, the SiO_{y} layer with 0.70≦y≦1.8 can be oxidized, or converted into an SiO₂ layer, with air or another oxygen-containing gas at a temperature of more than 200° C., preferably more than 400° C. and less than 600° C. For example, aluminum flakes coated with SiO_{y} (y=1) can be converted Into aluminum flakes coated with SiO_{z} (z=1.40 to 2.00) by heating at 500-600° C. for several hours in an oxygen-containing atmosphere. In that process, if the entire SiO_{y} is not converted into SiO₂, an SiO₂ layer forms on the surface of the SiO_{y} layer, with y gradually decreasing towards the aluminum layer.

A further preferred embodiment of the present invention therefore relates to aluminum flakes comprising

(C1) a layer consisting of SiO₂,

(A1) a layer consisting of SiO_{y} on the layer (C1),

(B) a layer consisting of aluminum on the layer (A1),

(A2) a layer consisting of SiO_{y} on the layer (B) and

(C2) a layer consisting of SiO₂ on the layer (A2), wherein 0.70≦y≦1.95, preferably 1.0≦y≦1.8, most preferably 1.4≦y≦1.8.

The layer thickness of the layer (B) consisting of aluminum is generally from 10 to 100 nm, preferably from 30 to 50 nm.

The layer thickness of the layers (A1) and (A2) consisting of SiO_{z}, the layer thickness of the layers (D1) and (D2) consisting of SiO₂, the layer thickness of the layer (A1) consisting of SiO_{y} and of the layer (C1) consisting of SiO₂ and the layer thickness of the layer (A2) consisting of SiO_{y} and of the layer (C2) consisting of SiO₂ is from 200 to 350 nm, preferably from 250 to 300 nm, when the aluminum flakes are the end product, and from 200 to 500 nm when the aluminum flakes are an intermediate for interference pigments. When the aluminum flakes are the end product, it is especially preferred for SiO_{y} with y about 1 to have a thickness of about 250±10 nm and for SiO₂ to have a thickness of about 300±10 nm.

The aluminum flakes according to the invention can be used in the applications that are customary for known aluminum flakes. Examples that may be mentioned are use of the aluminum flakes according to the invention in paints, electrostatic coatings, in ink-jet printing, cosmetics, coatings, printing inks, plastics materials, in glazes for ceramics and glass and in security printing.

It is also possible for the finished aluminum flakes to be subjected to after-coating or after-treatment, which further increases the stability to light, weathering and chemicals or facilitates handling of the pigment, especially incorporation into various media. The processes described in EP-A-477433, EP-A-826745 or EP-A-1084198, for example, are suitable as after-treatment or after-coating.

The aluminum flakes can furthermore be converted into interference pigments by being coated with further layers. The fundamental structure of such pigments is described, for example, in the following patents: EP-A-571836, EP-A-708154, EP-A-768343, EP-A-1025168 and WO00/34395.

In order to be able to use the aluminium flakes (flake-form aluminium) in aqueous compositions, it is necessary for those pigments to be protected against corrosion by water. According to R. Besold, Aluminiumpigmente für wässrige Beschichtungen-Widerspruch oder Wirklichkeit?, Farbe+Lack 97 (1991) 311-314, a large number of procedures, which can be divided into two groups, are known for the stabilisation of aluminium pigments:

adsorption of corrosion inhibitors on the pigment surface
phosphoric acid esters: DE-A-3020073, EP-A-170474, EP-A-133644, U.S. Pat. No. 4,565,716, U.S. Pat. No. 4,808,231,
phosphates and phosphates: U.S. Pat. No. 4,565,716, U.S. Pat. No. 4,808,231, EP-A-240367,
vanadates: EP-A-305560, EP-A-104075,
chromates: U.S. Pat. No. 2,904,523, U.S. Pat. No. 4,693,754, EP-A-259592,
dimeric acids: DE-A-3002175, and

encapsulation of the pigments with a continuous inorganic protective layer:
SiO₂: U.S. Pat. No. 2,885,366, U.S. Pat. No. 3,954,496,
Fe₂O₃: DE-A-3003352,
TiO₂: DE-A-3813335,

or organic protective layer:
DE-A-3630356, DE-A-3147177, EP-A-477433, especially resins modified with phosphoric add: EP-A-170474, CA-A-1,273,733, AT-A-372696, DE-A-3807588, EP-A-319971.

In an especially preferred embodiment, the interference pigments on the basis of the silicon oxide/metal substrate comprises a further layer of a dielectric material having a “high” refractive index, that is to say a refractive index greater than about 1.65, preferably greater than about 2.0, most preferred greater than about 2.2, which is applied to the entire surface of the silicon oxide/metal substrate. Examples of such a dielectric material are zinc sulfide (ZnS), zinc oxide (ZnO), zirconium oxide (ZrO₂), titanium dioxide (TiO₂), carbon, indium oxide (In₂O₃), indium tin oxide (ITO), tantalum pentoxide (Ta₂O₅), chromium oxide (Cr₂O₃), cerium oxide (CeO₂), yttrium oxide (Y₂O₃), europium oxide (Eu₂O₃), iron oxides such as iron(II)/iron(III) oxide (Fe₃O₄) and iron(III) oxide (Fe₂O₃), hafnium nitride (HfN), hafnium carbide (HfC), hafnium oxide (HfO₂), lanthanum oxide (La₂O₃), magnesium oxide (MgO), neodymium oxide (Nd₂O₃), praseodymium oxide (Pr₆O₁₁), samarium oxide (Sm₂O₃), antimony trioxide (Sb₂O₃), silicon monoxides (SiO), selenium trioxide (Se₂O₃), tin oxide (SnO₂), tungsten trioxide (WO₃), or combinations thereof. The dielectric material is preferably a metal oxide. It being possible for the metal oxide to be a single oxide or a mixture of oxides, with or without absorbing properties, for example, TiO₂, ZrO₂, Fe₂O₃, Fe₃O₄, Cr₂O₃, iron titanate, iron oxide hydrates, titanium suboxides, or ZnO, with TiO₂ being especially preferred. It is possible to obtain pigments that are more intense in color by applying, on top of the TiO₂ layer, a metal oxide of low refractive index. Nonlimiting examples of suitable low index dielectric materials that can be used include silicon dioxide (SiO₂), aluminum oxide (Al₂O₃), and metal fluorides such as magnesium fluoride (MgF₂), aluminum fluoride (AlF₃), cerium fluoride (CeF₃), lanthanum fluoride (LaF₃), sodium aluminum fluorides (e.g., Na₃AlF₆ or Na₅Al₃F₁₄), neodymium fluoride (NdF₃), samarium fluoride (SmF₃), barium fluoride (BaF₂), calcium fluoride (CaF₂), lithium fluoride (LiF), combinations thereof, or any other low index material having an index of refraction of about 1.65 or less. For example, organic monomers and polymers can be utilized as low index materials, including dienes or alkenes such as acrylates (e.g., methacrylate), polymers of perfluoroalkenes, polytetrafluoroethylene (TEFLON), polymers of fluorinated ethylene propylene (FEP), parylene, p-xylene, combinations thereof, and the like. Additionally, the foregoing materials include evaporated, condensed and cross-linked transparent acrylate layers, which may be deposited by methods described in U.S. Pat. No. 5,877,895, or EP-A-733,919, the disclosure of which is incorporated herein by reference. SiO₂, Al₂O₃, AlOOH, B₂O₃, or a mixture thereof, are preferred. SiO₂ is most preferred.

The metal oxide layers can be applied by CVD (chemical vapour deposition) or by wet chemical coating. The metal oxide layers can be obtained by decomposition of metal carbonyls in the presence of water vapour (relatively low molecular weight metal oxides such as magnetite) or in the presence of oxygen and, where appropriate, water vapour (e.g. nickel oxide and cobalt oxide). The metal oxide layers are especially applied by means of oxidative gaseous phase decomposition of metal carbonyls (e.g. iron pentacarbonyl, chromium hexacarbonyl; EP-A-45 851), by means of hydrolytic gaseous phase decomposition of metal alcoholates (e.g. titanium and zirconium tetra-n- and iso-propanolate; DE-A-41 40 900) or of metal halides (e.g. titanium tetrachloride; EP-A-338 428), by means of oxidative decomposition of organyl tin compounds (especially alkyl tin compounds such as tetrabutyltin and tetramethyltin; DE-A-44 03 678) or by means of the gaseous phase hydrolysis of organyl silicon compounds (especially di-tert-butoxyacetoxysilane) described in EP-A-668 329, it being possible for the coating operation to be carried out in a fluidised-bed reactor (EP-A-045 851 and EP-A-106 235).

Phosphate-, chromate- and/or vanadate-containing and also phosphate- and SiO₂-containing metal oxide layers can be applied in accordance with the passivation methods described in DE-A-42 36 332, EP-A-678 561 and in EP-A-826 745 by means of hydrolytic or oxidative gaseous phase decomposition of oxide-halides of the metals (e.g. CrO₂Cl₂, VOCl₃), especially of phosphorus oxyhalides (e.g. POCl₃), phosphoric and phosphorous acid esters (e.g. di- and tri-methyl and di- and tri-ethyl phosphite) and of amino-group-containing organyl silicon compounds (e.g. 3-aminopropyl-triethoxy- and -trimethoxy-silane).

Layers of oxides of the metals zirconium, titanium, iron and zinc, oxide hydrates of those metals, iron titanates, titanium suboxides or mixtures thereof are preferably applied by precipitation by a wet chemical method, it being possible, where appropriate, for the metal oxides to be reduced. In the case of the wet chemical coating, the wet chemical coating methods developed for the production of pearlescent pigments may be used; these are described, for example, in DE-A-14 67468, DE-A-19 59 988, DE-A-20 09 566, DE-A-22 14 545, DE-A-22 15 191, DE-A-22 44 298, DE-A-23 13 331, DE-A-25 22 572, DE-A-31 37 808, DE-A-31 37 809, DE-A-31 51 343, DE-A-31 51 354, DE-A-31 51 355, DE-A-32 11 602 and DE-A-32 35 017, DE 195 99 88, WO 93/08237, WO98/53001 and WO03/6558.

The metal oxide of high refractive index is preferably TiO₂ and/or iron oxide, and the metal oxide of low refractive index is preferably SiO₂. Layers of TiO₂ can be in the rutile or anastase modification, wherein the ruble modification is preferred. TiO₂ layers can also be reduced by known means, for example ammonia, hydrogen, hydrocarbon vapor or mixtures thereof, or metal powders, as described in EP-A-735,114, DE-A-3433657, DE-A-4125134, EP-A-332071, EP-A-707,050 or WO93/19131.

For the purpose of coating, the substrate particles are suspended in water and one or more hydrolysable metal salts are added at a pH suitable for the hydrolysis, which is so selected that the metal oxides or metal oxide hydrates are precipitated directly onto the particles without subsidiary precipitation occurring. The pH is usually kept constant by simultaneously metering in a base. The pigments are then separated off, washed, dried and, where appropriate, calcinated, it being possible to optimise the calcinating temperature with respect to the coating in question. If desired, after individual coatings have been applied, the pigments can be separated off, dried and, where appropriate, calcinated, and then again re-suspended for the purpose of precipitating further layers.

The metal oxide layers are also obtainable, for example, in analogy to a method described in DE-A-195 01 307, by producing the metal oxide layer by controlled hydrolysis of one or more metal acid esters, where appropriate in the presence of an organic solvent and a basic catalyst, by means of a sol-gel process. Suitable basic catalysts are, for example, amines, such as triethylamine, ethylenediamine, tributylamine, dimethylethanolamine and methoxy-propylamine. The organic solvent is a water-miscible organic solvent such as a C₁₋₄alcohol, especially isopropanol.

Suitable metal acid esters are selected from alkyl and aryl alcoholates, carboxylates, and carboxyl-radical- or alkyl-radical- or aryl-radical-substituted alkyl alcoholates or carboxylates of vanadium, titanium, zirconium, silicon, aluminium and boron. The use of triisopropyl aluminate, tetraisopropyl titanate, tetraisopropyl zirconate, tetraethyl orthosilicate and triethyl borate is preferred. In addition, acetylacetonates and acetoacetylacetonates of the aforementioned metals may be used. Preferred examples of that type of metal acid ester are zirconium acetylacetonate, aluminium acetylacetonate, titanium acetylacetonate and diisobutyloleyl acetoacetylaluminate or diisopropyloleyl acetoacetylacetonate and mixtures of metal acid esters, for example Dynasil® (Hüls), a mixed aluminium/silicon metal acid ester.

As a metal oxide having a high refractive index, titanium dioxide is preferably used, the method described in U.S. Pat. No. 3,553,001 being used, in accordance with an embodiment of the present invention, for application of the titanium dioxide layers.

An aqueous titanium salt solution is slowly added to a suspension of the material being coated, which suspension has been heated to about 50-100° C., especially 70-80° C., and a substantially constant pH value of about from 0.5 to 5, especially about from 1.2 to 2.5, is maintained by simultaneously metering in a base such as, for example, aqueous ammonia solution or aqueous alkali metal hydroxide solution. As soon as the desired layer thickness of precipitated TiO₂ has been achieved, the addition of titanium salt solution and base is stopped.

This method, also referred to as a titration method, is distinguished by the fact that an excess of titanium salt is avoided. That is achieved by feeding in for hydrolysis, per unit time, only that amount which is necessary for even coating with the hydrated TiO₂ and which can be taken up per unit time by the available surface of the particles being coated. In principle, the anatase form of TiO₂ forms on the surface of the starting pigment. By adding small amounts of SnO₂, however, it is possible to force the rutile structure to be formed.

The thickness of the TiO₂ layer is generally in the range of from 5 to 100 nm, especially 10 to 25 nm.

The TiO₂ can optionally be reduced by usual procedures: U.S. Pat. No. 4,948,631 (NH₃, 750-850° C.), WO93/19131 (H₂,>900° C.) or DE-A-19843014 (solid reduction agent, such as, for example, silicon, >600° C.).

Where appropriate, an SiO₂ (protective) layer can be applied on top of the titanium dioxide layer, for which the following method may be used: A soda waterglass solution is metered in to a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

The interference pigments are preferably obtained by suspending the SiO₂- or SiO_{y}-coated aluminum flakes in water and coating with a metal oxide hydrate having a high refractive index and, where appropriate, a metal oxide hydrate of low refractive index by addition and hydrolysis of corresponding, water-soluble metal compounds by means of conventional processes, the pH value required for precipitation of the metal oxide hydrate in question being established and kept constant by the simultaneous addition of acid or base, and subsequently separating the coated carrier material from the aqueous suspension, drying and, where appropriate, calcining (see, for example, DE 1959988, DE 2215191, DE2244298, DE2313331, DE2522572, DE 3137808, DE3137809, DE 3151343, DE3151355, DE3211602, DE3235017, WO93/08237 and WO98/53011).

Instead of a layer of a metal oxide having a high Index of refraction U.S. Pat. No. 6,524,381 materials, such as diamond-like carbon and amorphous carbon, can be deposited by plasma-assisted vacuum methods (using vibrating conveyors, rotating drum coaters, oscillatory drum coaters, and free-fall chambers) as described, for example in U.S. Pat. No. 6,524,381, on the SiO_{z}-coated metal substrates.

Consequently, the present invention also relates to plane-parallel structures (pigments) based on silicon oxide/aluminum substrates having on their surface a carbon layer especially a diamond-like carbon layer having a thickness of 5 to 150 nm, especially 20 to 70 nm, more especially 30 to 70 nm.

In the method described, for example, in U.S. Pat. No. 6,015,597, diamond-like network (DLN) coatings are deposited onto particles from carbon-containing gases, such as, for example, acetylene, methane, butadiene and mixtures of these and optionally Ar, and optionally gases containing additional components by plasma deposition. Deposition occurs at reduced pressures (relative to atmospheric pressure) and in a controlled environment. A carbon rich plasma is created in a reaction chamber by applying an electric field to a carbon-containing gas. Particles to be coated are held in a vessel or container in the reactor and are agitated while in proximity to the plasma. Species within the plasma react on the particle surface to form covalent bonds, resulting in DLN on the surface of the particles.

The term “diamond-like network” (DLN) refers to amorphous films or coatings comprised of carbon and optionally comprising one or more additional components selected from the group consisting of hydrogen, nitrogen, oxygen, fluorine, silicon, sulfur, titanium, and copper. The diamond-like networks comprise approximately 30 to 100 atomic percent carbon, with optional additional components making up the remainder

Coating of the SiO_{z}-coated flakes with a diamond-like carbon layer or a diamond-like network can be carried out, for example, by a process and an apparatus described in EP-A-1034320, comprising

providing a capacitively coupled reactor system, comprising two electrodes in an evacuable reaction chamber,

placing a multiplicity of particles in proximity to one of the electrodes;

evacuating the chamber;

allowing high-frequency energy to act on the electrode that is in proximity to the particles and grounding the other electrode,

introducing a carbon-containing source, for example hydrocarbon gases, such as acetylene, methane, butadiene or mixtures thereof, into the reaction chamber thereby forming a plasma comprising reactive species in proximity to the multiplicity of particles,

and further forming an Ion cloud around the grounded electrode,

agitating the flakes in such a manner as to expose the surfaces thereof to the reactive species in the plasma while keeping the particles substantially within the ion cloud.

The diamond-like network may consist of carbon and, where applicable, may comprise one or more components consisting of hydrogen, nitrogen, oxygen, fluorine, silicon, sulfur, titanium or copper.

The present invention therefore relates also to pigments based on the aluminum flakes according to the invention comprising on the layers (A1) and (A2) or on the layers (C1) and (C2), preferably over the entire surface of the aluminum flakes, a layer (E) consisting of a layer of a dielectric material having a “high” refractive index, especially TiO₂, or of carbon, preferably diamond-like carbon, and

to pigments based on the aluminum flakes according to the invention comprising on the layers (D1) and (D2), preferably over the entire surface of the aluminum flakes, a layer (E) consisting of a dielectric material having a “high” refractive index, especially TiO₂, or of carbon, preferably diamond-like carbon.

The thickness of the carbon layer is generally from 5 to 150 nm, preferably from 30 to 70 nm.

Furthermore, the SiO_{z}-coated aluminum flakes may, as described in EP-A-0 982 376, be coated with a nitrogen-doped carbon layer. The process described in EP-A-0 982 376 comprises the following steps:

(a) suspending the silicon oxide-coated aluminum flakes in a liquid,

(b) where appropriate adding a surface-modifier and/or a polymerization catalyst,

(c), before or after step (b), adding one or more polymers comprising nitrogen and carbon atoms, or one or more monomers capable of forming such polymers,

(d) forming a polymeric coating on the surface of the flakes,

(e) Isolating the coated flakes and

(f) heating the coated flakes to a temperature of from 100 to 600° C. in a gaseous atmosphere.

The polymer may be a polypyrrole, a polyamide, a polyaniline, a polyurethane, a nitrile rubber or a melamine-formaldehyde resin, preferably a polyacrylonitrile, or the monomer is a pyrrole derivative, an acrylonitrile, a methacrylonitrile, a crotonitrile, an acrylamide, a methacrylamide or a crotonamide, preferably an acrylonitrile, methacrylonitrile or crotonitrile, most preferably an acrylonitrile.

Preferably, the flakes are heated in step (f) initially to from 100° C. to 300° C. in an oxygen-containing atmosphere and then to from 200 to 600° C. in an inert gas atmosphere.

The present invention therefore relates also to pigments based on the aluminum flakes according to the invention comprising over the entire surface of the silicon oxide coated aluminum flakes a layer (F) consisting of from 50 to 95% by weight carbon, from 5 to 25% by weight nitrogen and from 0 to 25% by weight of the elements hydrogen, oxygen and/or sulfur, the percentage by weight data relating to the total weight of the layer (F).

The thickness of the nitrogen-doped carbon layer is generally from 10 to 150 nm, preferably from 30 to 70 nm.

The invention also relates to the use of the pigments based on the aluminum flakes according to the invention in paints, textiles (see, for example, PCT/EP03/11188), ink-jet printing (see, for example, PCT/EP03/50690), cosmetics (see, for example, PCT/EP03/09269), printing inks, plastics materials, coatings, especially in automotive finishes, in glazes for ceramics and glass, and in security printing.

If the layers present on the aluminum core are applied by PVD, those layers will be present only on the parallel faces of the core and not, however, on the side faces. If (further) layers are applied by wet-chemical precipitation, they will cover the entire surface of the flakes.

The SiO_{y}-coated aluminum flakes can fundamentally be obtained by means of a process comprising the following steps (U.S. Pat. No. 6,270,840, WO00/18978, WO02/090613, WO03/90613):

a) vapor-deposition of a separating agent onto a carrier to produce a separating agent layer,

b) vapor-deposition of an SiO_{y} layer onto the separating agent layer,

c) vapor-deposition of an Al layer onto the SiO_{y} layer,

d) vapor-deposition of an SiO_{y} layer onto the Al layer,

e) dissolution of the separating agent layer in a solvent and

f) separation of the SiO_{y}-coated aluminum flakes from the solvent, wherein 0.70≦y≦1.95, preferably 1.0≦y≦1.80, most preferably 1.10≦y≦1.80.

The silicon oxide layer (SiO_{y}) is formed preferably from silicon monoxide vapor produced in the vaporizer by reaction of a mixture of Si and SiO₂ at temperatures of more than 1300° C. A SiO_{y} layer with 0.70≦y≦0.99 is formed preferably by evaporating silicon monoxide containing silicon in an amount up to 20% by weight at temperatures of more than 1300° C.

The above-mentioned process makes available silicon oxide coated aluminum flakes having a high plane-parallelism and a defined thickness in the range of ±10%, preferably ±5%, of the average thickness, and low reflection.

The SiO_{y} layers are obtained by heating a preferably stoichiometric mixture of fine silicon and quartz (SiO₂) powder in a vaporizer described, for example, in DE 43 42 574 C1 and in U.S. Pat. No. 6,202,591 to more than 1300° C. under a high vacuum. The reaction product is silicon monoxide gas, which under vacuum is directed directly onto the passing carrier, where it condenses as SiO. Non-stoichiometric mixtures may also be used. The vaporizer contains a charge comprising a mixture of Si and SiO₂, SiO_{y}, or a mixture thereof, the particle size of the substances that react with one another (Si and SiO₂) being advantageously less than 0.3 mm. The weight ratio of Si to SiO₂ is advantageously in the range from 0.15:1 to 0.75:1 (parts by weight); preferably, a stoichiometric mixture is present SiO_{y} present in the vaporizer vaporizes directly. Si and SiO₂ react at a temperature of more than 1300° C. to form silicon monoxide vapor. The separating agent condensed onto the carrier may be a laquer, a thermoplastic polymer described in U.S. Pat. No. 6,398,999, such as acrylic or styrene polymers or mixtures thereof, an organic substance soluble In organic solvents or water and vaporizable in vacuo, such as anthracene, anthraquinone, acetamidophenol, acetylsalicylic acid, camphoric anhydride, benzimidazole, benzene-1,2,4-tricarboxylic add, biphenyl-2,2-dicarboxylic acid, bis(4-hydroxyphenyl)sulfone, dihydroxyanthraquinone, hydantoin, 3-hydroxybenzoic acid, 8-hydroxyquinoline-5-sulfonic acid monohydrate, 4-hydroxycoumarin, 7-hydroxycoumarin, 3-hydroxynaphthalene-2-carboxylic acid, isophthalic acid, 4,4-methylene-bis-3-hydroxynaphthalene-2-carboxylic acid, naphthalene-1,8-dicarboxylic anhydride, phthalimide and its potassium salt, phenolphthalein, phenothiazine, saccharin and its salts, tetraphenylmethane, triphenylene, triphenylmethanol or a mixture of at least two of those substances. The separating agent is preferably an inorganic salt soluble in water and vaporizable in vacuo (see, for example, DE 198 44 357), such as sodium chloride, potassium chloride, lithium chloride, sodium fluoride, potassium fluoride, lithium fluoride, calcium fluoride, sodium aluminum fluoride and disodium tetraborate.

Step e) is usually carried out at a pressure that is higher than the pressure in steps a) and b) and lower than atmospheric pressure.

The (movable) carrier preferably comprises one or more continuous metal belts, with or without a polymer coating, or one or more polyimide or polyethylene terephthalate belts. The movable carrier may furthermore comprise one or more discs, cylinders or other rotationally symmetrical bodies, which rotate about an axis.

The SiO_{y}-coated aluminum flakes are separated from the solvent of the separating agent preferably by washing-out and subsequent filtration, sedimentation, centrifugation, decanting or evaporation. Furthermore, the SiO_{y}-coated aluminum flakes may, after washing-out of the dissolved separating agent contained in the solvent, be frozen together with the solvent and subsequently subjected to a process of freeze-drying, during which the solvent is separated off as a result of sublimation below the triple point and the dry flakes remain behind in the form of individual plane-parallel structures.

The condensed silicon suboxide corresponds to the formula SiO_{y}, with 0.95≦y≦1.8, preferably with about 1.0≦y≦1.5, y values of less than 1 being obtained by means of an excess of silicon in the vaporizer material. Except under an ultra-high vacuum, in industrial vacuums of a few 10⁻² Pa vaporized SiO always condenses as SiO_{y} with 1≦y≦1.8, especially with 1.1≦y≦1.5, because high-vacuum apparatuses always contain, as a result of gas emission from surfaces, traces of water vapor which react with the readily reactive SiO at vaporization temperature.

In detail, a salt, for example NaCl, followed by layers of silicon suboxide (SiO_{y}), Al and SiO_{y} are successively vapor-deposited onto a carrier, which may be a continuous metal belt, passing by way of the vaporizers under a vacuum of <0.5 Pa. The vapor-deposited thicknesses of salt are approximately from 20 to 100 nm, preferably from 30 to 60 nm, those of SiO are, depending on the intended use of the product, from 200 to 500 nm, and those of aluminum are from 10 to 100 nm. On its further course, the belt-form carrier, which is closed to form a loop, runs through dynamic vacuum lock chambers of known construction (cf. U.S. Pat. No. 6,270,840) into a region of from 1 to 5×10⁴ Pa pressure, preferably from 600 to 10⁹ Pa pressure, and especially from 10³ to 5×10³ Pa pressure, where it is immersed in a separating bath. The temperature of the solvent should be so selected that its vapor pressure is in the indicated pressure range. With mechanical assistance, the separating agent layer rapidly dissolves and the product layer breaks up into flakes, which are then in the form of a suspension in the solvent. On its further course, the belt is dried and freed from any contaminants still adhering to it. It runs through a second group of dynamic vacuum lock chambers back into the vaporization chamber, where the process of coating with separating agent and product layer is repeated.

The suspension then obtained in both cases, comprising product structures and solvent with separating agent dissolved therein, is then separated in a further operation in accordance with a known technique. For that purpose, the product structure is first concentrated in the liquid and rinsed several times with fresh solvent in order to wash out the dissolved separating agent. The product, in the form of a solid that is still wet, is then separated off by means of filtration, sedimentation, centrifugation, decanting or evaporation, and is dried.

Separating off the plane-parallel structures after washing-out at atmospheric pressure can be carried out under gentle conditions by freezing the suspension, which has been concentrated to a solids content of approximately 50%, and subjecting it in known manner to freeze-drying at approximately −10° C. and 50 Pa pressure. The dry substance remains behind as product, which can be subjected to the steps of further processing by means of coating or chemical conversion.

Instead of using a continuous belt, it is possible to produce the product using a rotary body by carrying out the steps of vapor-deposition of separating agent and SiO_{y}, Al and SiO_{y}, of separation, and of drying the carrier in accordance with DE-A-199 52 032. The rotary body may be one or more discs, a cylinder or any other rotationally symmetrical body.

The flakes can then be subjected to oxidative heat treatment. For example, air or some other oxygen-containing gas is passed through the platelets, which are in the form of loose material or in a fluidized bed, at a temperature of more than 200° C., preferably more than 400° C. and especially from 400 to 600° C.

Comminution of the fragments of film to pigment size can be effected, for example, by means of ultrasound or by mechanical means using high-speed stirrers in a liquid medium, or after drying the fragments in an air-jet mill having a rotary classifer. Depending on whether the pigment comminution is carried out in a liquid medium or in the dry state, passivation of the free metal surfaces of the aluminium pigment is carried out either during the comminution procedure, or following that procedure, by means of one of the above-mentioned processes.

It is also possible for the aluminum flakes or pigments to be subjected to after-coating or after-treatment, which further increases stability to light, weathering and chemicals or facilitates handling of the pigment, especially incorporation into various media. The processes described in EP-A-1084198, EP-A-826745, DE-A-22 15 191, DE-A-31 51 354, DE-A-32 35 017 or DE-A-33 34 598, for example, are suitable as after-treatment or after-coating.

Where appropriate, an SiO₂ protective layer can be applied, for which the following method may be used: a soda waterglass solution is metered into a suspension of the material being coated, which suspension has been heated to about 50-100° C., especially 70-80° C. The pH is maintained at from 4 to 10, preferably from 6.5 to 8.5, by simultaneously adding 10% hydrochloric acid. After addition of the waterglass solution, stirring is carried out for 30 minutes.

The pigments according to the invention are distinguished by having a very uniform thickness, as a result of which very high color purity and color strength are obtained. Metallic or non-metallic, inorganic platelet-shaped particles or pigments are effect pigments, (especially metal effect pigments or interference pigments), that is to say, pigments that, besides imparting color to an application medium, impart additional properties, for example angle dependency of the color (flop), lustre (not surface gloss) or texture. On metal effect pigments (aluminum flakes), substantially oriented reflection occurs at directionally oriented pigment particles. In the case of interference pigments (pigments), the color-imparting effect is due to the phenomenon of interference of light in thin, highly refractive layers.

The pigments according to the invention can be used for all customary purposes, for example for coloring polymers in the mass, coatings (including effect finishes, including those for the automotive sector) and printing inks (including offset printing, intaglio printing, bronzing and flexographic printing), and also, for example, for applications in cosmetics, in ink-jet printing, for dyeing textiles, glazes for ceramics and glass as well as laser marking of papers and plastics. Such applications are known from reference works, for example “Industrielle Organische Pigmente” (W. Herbst and K. Hunger, VCH Verlagsgesellschaft mbH, Weinheim/N.Y., 2nd, completely revised edition, 1995).

When the pigments according to the invention are interference pigments (effect pigments), they are goniochromatic and result in brilliant, highly saturated (lustrous) colors. They are accordingly very especially suitable for combination with conventional, transparent pigments, for example organic pigments such as, for example, diketopyrrolopyrroles, quinacridones, dioxazines, perylenes, isoindolinones etc., it being possible for the transparent pigment to have a similar color to the effect pigment. Especially interesting combination effects are obtained, however, in analogy to, for example, EP-A-388 932 or EP-A-402 943, when the color of the transparent pigment and that of the effect pigment are complementary.

The pigments according to the invention can be used with excellent results for pigmenting high molecular weight organic material.

The high molecular weight organic material for the pigmenting of which the pigments or pigment compositions according to the invention may be used may be of natural or synthetic origin. High molecular weight organic materials usually have molecular weights of about from 10³ to 10⁸ g/mol or even more. They may be, for example, natural resins, drying oils, rubber or casein, or natural substances derived therefrom, such as chlorinated rubber, oil-modified alkyd resins, viscose, cellulose ethers or esters, such as ethylcellulose, cellulose acetate, cellulose propionate, cellulose acetobutyrate or nitrocellulose, but especially totally synthetic organic polymers (thermosetting plastics and thermoplastics), as are obtained by polymerisation, polycondensation or polyaddition. From the class of the polymerisation resins there may be mentioned, especially, polyolefins, such as polyethylene, polypropylene or polyisobutylene, and also substituted polyolefins, such as polymerisation products of vinyl chloride, vinyl acetate, styrene, acrylonitrile, acrylic acid esters, methacrylic acid esters or butadiene, and also copolymerisation products of the said monomers, such as especially ABS or EVA.

From the series of the polyaddition resins and polycondensation resins there may be mentioned, for example, condensation products of formaldehyde with phenols, so-called phenoplasts, and condensation products of formaldehyde with urea, thiourea or melamine, so-called aminoplasts, and the polyesters used as surface-coating resins, either saturated, such as alkyd resins, or unsaturated, such as maleate resins; also linear polyesters and polyamides, polyurethanes or silicones.

The said high molecular weight compounds may be present singly or in mixtures, in the form of plastic masses or melts. They may also be present in the form of their monomers or in the polymerised state in dissolved form as film-formers or binders for coatings or printing inks, such as, for example, boiled linseed oil, nitrocellulose, alkyd resins, melamine resins and urea-formaldehyde resins or acrylic resins.

Depending on the intended purpose, it has proved advantageous to use the (effect) pigments or (effect) pigment compositions according to the invention as toners or in the form of preparations. Depending on the conditioning method or intended application, it may be advantageous to add certain amounts of texture-improving agents to the effect pigment before or after the conditioning process, provided that this has no adverse effect on use of the effect pigments for coloring high molecular weight organic materials, especially polyethylene. Suitable agents are, especially, fatty acids containing at least 18 carbon atoms, for example stearic or behenic acid, or amides or metal salts thereof, especially magnesium salts, and also plasticisers, waxes, resin acids, such as abietic acid, rosin soap, alkylphenols or aliphatic alcohols, such as stearyl alcohol, or aliphatic 1,2-dihydroxy compounds containing from 8 to 22 carbon atoms, such as 1,2-dodecanediol, and also modified colophonium maleate resins or fumaric add colophonium resins. The texture-improving agents are added in amounts of preferably from 0.1 to 30% by weight, especially from 2 to 15% by weight, based on the end product.

The (effect) pigments according to the invention can be added In any tinctorially effective amount to the high molecular weight organic material being pigmented. A pigmented composition comprising a high molecular weight organic material and from 0.01 to 80% by weight, preferably from 0.1 to 30% by weight, based on the high molecular weight organic material, of an pigment according to the invention is advantageous. Concentrations of from 1 to 20% by weight, especially of about 10% by weight, can often be used in practice.

High concentrations, for example those above 30% by weight, are usually in the form of concentrates (“masterbatches”) which can be used as colorants for producing pigmented materials having a relatively low pigment content, the pigments according to the invention having an extraordinarily low viscosity in customary formulations so that they can still be processed well.

For the purpose of pigmenting organic materials, the effect pigments according to the invention may be used singly. It is, however, also possible, in order to achieve different hues or color effects, to add any desired amounts of other color-imparting constituents, such as white, colored, black or effect pigments, to the high molecular weight organic substances in addition to the effect pigments according to the invention. When colored pigments are used in admixture with the effect pigments according to the invention, the total amount is preferably from 0.1 to 10% by weight, based on the high molecular weight organic material. Especially high goniochromicity is provided by the preferred combination of an effect pigment according to the invention with a colored pigment of another color, especially of a complementary color, with colorations made using the effect pigment and colorations made using the colored pigment having, at a measurement angle of 10°, a difference in hue (ΔH*) of from 20 to 340, especially from 150 to 210.

Preferably, the effect pigments according to the invention are combined with transparent colored pigments, it being possible for the transparent colored pigments to be present either in the same medium as the effect pigments according to the invention or in a neighbouring medium. An example of an arrangement in which the effect pigment and the colored pigment are advantageously present in neighbouring media is a multi-layer effect coating.

The pigmenting of high molecular weight organic substances with the pigments according to the invention is carried out, for example, by admixing such a pigment, where appropriate in the form of a masterbatch, with the substrates using roll mills or mixing or grinding apparatuses. The pigmented material is then brought into the desired final form using methods known per se, such as calendering, compression moulding, extrusion, coating, pouring or injection moulding. Any additives customary in the plastics industry, such as plasticisers, fillers or stabilisers, can be added to the polymer, in customary amounts, before or after incorporation of the pigment. In particular, in order to produce non-rigid shaped articles or to reduce their brittleness, It is desirable to add plasticisers, for example esters of phosphoric acid, phthalic add or sebacic acid, to the high molecular weight compounds prior to shaping.

For pigmenting coatings and printing inks, the high molecular weight organic materials and the effect pigments according to the invention, where appropriate together with customary additives such as, for example, filers, other pigments, siccatives or plasticisers, are finely dispersed or dissolved in the same organic solvent or solvent mixture, it being possible for the individual components to be dissolved or dispersed separately or for a number of components to be dissolved or dispersed together, and only thereafter for all the components to be brought together.

Dispersing an effect pigment according to the invention in the high molecular weight organic material being pigmented, and processing a pigment composition according to the invention, are preferably carried out subject to conditions under which only relatively weak shear forces occur so that the effect pigment is not broken up into smaller portions.

Plastics comprise the pigment of the invention in amounts of 0.1 to 50% by weight, in particular 0.5 to 7% by weight. In the coating sector, the pigments of the invention are employed in amounts of 0.1 to 10% by weight. In the pigmentation of binder systems, for example for paints and printing inks for intaglio, offset or screen printing, the pigment is incorporated into the printing ink in amounts of 0.1 to 50% by weight, preferably 5 to 30% by weight and in particular 8 to 15% by weight.

The colorations obtained, for example in plastics, coatings or printing inks, especially in coatings or printing inks, more especially in coatings, are distinguished by excellent properties, especially by extremely high saturation, outstanding fastness properties and high goniochromicity.

When the high molecular weight material being pigmented is a coating, it is especially a speciality coating, very especially an automotive finish.

The effect pigments according to the invention are also suitable for making-up the lips or the skin and for coloring the hair or the nails.

The invention accordingly relates also to a cosmetic preparation or formulation comprising from 0.0001 to 90% by weight of a pigment, especially an effect pigment, according to the invention and from 10 to 99.9999% of a cosmetically suitable carrier material, based on the total weight of the cosmetic preparation or formulation.

Such cosmetic preparations or formulations are, for example, lipsticks, blushers, foundations, nail varnishes and hair shampoos.

The pigments may be used singly or in the form of mixtures. It is, in addition, possible to use pigments according to the invention together with other pigments and/or colorants, for example in combinations as described hereinbefore or as known in cosmetic preparations. The cosmetic preparations and formulations according to the invention preferably contain the pigment according to the invention in an amount from 0.005 to 50% by weight, based on the total weight of the preparation.

Suitable carrier materials for the cosmetic preparations and formulations according to the invention include the customary materials used in such compositions.

The cosmetic preparations and formulations according to the invention may be in the form of, for example, sticks, ointments, creams, emulsions, suspensions, dispersions, powders or solutions. They are, for example, lipsticks, mascara preparations, blushers, eye-shadows, foundations, eyeliners, powder or nail varnishes.

If the preparations are in the form of sticks, for example lipsticks, eye-shadows, blushers or foundations, the preparations consist for a considerable part of fatty components, which may consist of one or more waxes, for example ozokerite, lanolin, lanolin alcohol, hydrogenated lanolin, acetylated lanolin, lanolin wax, beeswax, candelilla wax, microcrystalline wax, carnauba wax, cetyl alcohol, stearyl alcohol, cocoa butter, lanolin fatty acids, petrolatum, petroleum jelly, mono-, di- or tri-glycerides or fatty esters thereof that are solid at 25° C., silicone waxes, such as methyloctadecane-oxypolysiloxane and poly(dimethylsiloxy)-stearoxysiloxane, stearic acid monoethanolamine, colophane and derivatives thereof, such as glycol abietates and glycerol abietates, hydrogenated oils that are solid at 25° C., sugar glycerides and oleates, myristates, lanolates, stearates and dihydroxystearates of calcium, magnesium, zirconium and aluminum.

The fatty component may also consist of a mixture of at least one wax and at least one oil, in which case the following oils, for example, are suitable: paraffin oil, purcelline oil, perhydrosqualene, sweet almond oil, avocado oil, calophyllum oil, castor oil, sesame oil, jojoba oil, mineral oils having a boiling point of about from 310 to 410° C., silicone oils, such as dimethylpolysiloxane, linoleyl alcohol, linolenyl alcohol, oleyl alcohol, cereal grain oils, such as wheatgerm oil, isopropyl lanolate, isopropyl palmitate, isopropyl myristate, butyl myristate, cetyl myristate, hexadecyl stearate, butyl stearate, decyl oleate, acetyl glycerides, octanoates and decanoates of alcohols and polyalcohols, for example of glycol and glycerol, ricinoleates of alcohols and polyalcohols, for example of cetyl alcohol, isostearyl alcohol, isocetyl lanolate, isopropyl adipate, hexyl laurate and octyl dodecanol.

The fatty components in such preparations in the form of sticks may generally constitute up to 99.91% by weight of the total weight of the preparation.

The cosmetic preparations and formulations according to the invention may additionally comprise further constituents, such as, for example, glycols, polyethylene glycols, polypropylene glycols, monoalkanolamides, non-coloured polymeric, inorganic or organic fillers, preservatives, UV filters or other adjuvants and additives customary in cosmetics, for example a natural or synthetic or partially synthetic di- or tri-glyceride, a mineral oil, a silicone oil, a wax, a fatty alcohol, a Guerbet alcohol or ester thereof, a lipophilic functional cosmetic active ingredient, including sun-protection filters, or a mixture of such substances. A lipophilic functional cosmetic active ingredient suitable for skin cosmetics, an active ingredient composition or an active ingredient extract is an ingredient or a mixture of ingredients that is approved for dermal or topical application. The following may be mentioned by way of example:

active ingredients having a cleansing action on the skin surface and the hair; these include all substances that serve to cleanse the skin, such as oils, soaps, synthetic detergents and solid substances;

active ingredients having a deodorising and perspiration-inhibiting action: they include antiperspirants based on aluminium salts or zinc salts, deodorants comprising bactericidal or bacteriostatic deodorising substances, for example triclosan, hexachlorophene, alcohols and cationic substances, such as, for example, quaternary ammonium salts, and odour absorbers, for example ®Grillocin (combination of zinc ricinoleate and various additives) or triethyl citrate (optionally in combination with an antioxidant, such as, for example, butyl hydroxytoluene) or ion-exchange resins;

active ingredients that offer protection against sunlight (UV filters): suitable active ingredients are filter substances (sunscreens) that are able to absorb UV radiation from sunlight and convert it into heat; depending on the desired action, the following light-protection agents are preferred: light-protection agents that selectively absorb sunburn-causing high-energy UV radiation in the range of approximately from 280 to 315 nm (UV-B absorbers) and transmit the longer-wavelength range of, for example, from 315 to 400 nm (UV-A range), as well as light-protection agents that absorb only the longer-wavelength radiation of the UV-A range of from 315 to 400 nm (UV-A absorbers); suitable light-protection agents are, for example, organic UV absorbers from the class of the p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, phenyl-benzimidazolesulfonic add and salts thereof, menthyl anthranilates, benzotriazole derivatives, and/or an inorganic micropigment selected from aluminium oxide- or silicon dioxide-coated TiO₂, zinc oxide or mica;

active ingredients against insects (repellents) are agents that are intended to prevent Insects from touching the skin and becoming active there; they drive insects away and evaporate slowly; the most frequently used repellent is diethyl toluamide (DEET); other common repellents will be found, for example, in “Pflegekosmetik” (W. Raab and U. Kindl, Gustav-Fischer-Verlag Stuttgart/N.Y., 1991) on page 161;

active ingredients for protection against chemical and mechanical influences: these include all substances that form a barrier between the skin and external harmful substances, such as, for example, paraffin oils, silicone oils, vegetable oils, PCL products and lanolin for protection against aqueous solutions, film-forming agents, such as sodium alginate, triethanolamine alginate, polyacrylates, polyvinyl alcohol or cellulose ethers for protection against the effect of organic solvents, or substances based on mineral oils, vegetable oils or silicone oils as “lubricants” for protection against severe mechanical stresses on the skin;

moisturising substances: the following substances, for example, are used as moisture-controlling agents (moisturisers): sodium lactate, urea, alcohols, sorbitol, glycerol, propylene glycol, collagen, elastin and hyaluronic acid;

active ingredients having a keratoplastic effect: benzoyl peroxide, retinoic acid, colloidal sulfur and resorcinol;

antimicrobial agents, such as, for example, triclosan or quaternary ammonium compounds;

oily or oil-soluble vitamins or vitamin derivatives that can be applied dermally: for example vitamin A (retinol in the form of the free acid or derivatives thereof), panthenol, pantothenic acid, folic acid, and combinations thereof, vitamin E (tocopherol), vitamin F; essential fatty acids; or niacinamide (nicotinic acid amide);

vitamin-based placenta extracts: active ingredient compositions comprising especially vitamins A, C, E, B₁, B₂, B₆, B₁₂, folic acid and biotin, amino adds and enzymes as well as compounds of the trace elements magnesium, silicon, phosphorus, calcium, manganese, iron or copper;

skin repair complexes: obtainable from inactivated and disintegrated cultures of bacteria of the bifidus group;

plants and plant extracts: for example amica, aloe, beard lichen, ivy, stinging nettle, ginseng, henna, camomile, marigold, rosemary, sage, horsetail or thyme;

animal extracts: for example royal jelly, propolis, proteins or thymus extracts;

cosmetic oils that can be applied dermally: neutral oils of the Miglyol 812 type, apricot kernel oil, avocado oil, babassu oil, cottonseed oil, borage oil, thistle oil, groundnut oil, gamma-oryzanol, rosehip-seed oil, hemp oil, hazelnut oil, blackcurrant-seed oil, jojoba oil, cherry-stone oil, salmon oil, linseed oil, cornseed oil, macadamia nut oil, almond oil, evening primrose oil, mink oil, olive oil, pecan nut oil, peach kernel oil, pistachio nut oil, rape oil, riceseed oil, castor oil, safflower oil, sesame oil, soybean oil, sunflower oil, tea tree oil, grapeseed oil or wheatgerm oil.

The preparations in stick form are preferably anhydrous but may in certain cases comprise a certain amount of water which, however, in general does not exceed 40% by weight, based on the total weight of the cosmetic preparation.

If the cosmetic preparations and formulations according to the invention are in the form of semi-solid products, that is to say in the form of ointments or creams, they may likewise be anhydrous or aqueous. Such preparations and formulations are, for example, mascaras, eyeliners, foundations, blushers, eye-shadows, or compositions for treating rings under the eyes.

If, on the other hand, such ointments or creams are aqueous, they are especially emulsions of the water-in-oil type or of the oil-in-water type that comprise, apart from the pigment, from 1 to 98.8% by weight of the fatty phase, from 1 to 98.8% by weight of the aqueous phase and from 0.2 to 30% by weight of an emulsifier.

Such ointments and creams may also comprise further conventional additives, such as, for example, perfumes, antioxidants, preservatives, gel-forming agents, UV filters, colorants, pigments, pearlescent agents, non-coloured polymers as well as inorganic or organic fillers. If the preparations are in the form of a powder, they consist substantially of a mineral or inorganic or organic filler such as, for example, talcum, kaolin, starch, polyethylene powder or polyamide powder, as well as adjuvants such as binders, colorants etc..

Such preparations may likewise comprise various adjuvants conventionally employed in cosmetics, such as fragrances, antioxidants, preservatives etc..

If the cosmetic preparations and formulations according to the invention are nail varnishes, they consist essentially of nitrocellulose and a natural or synthetic polymer in the form of a solution in a solvent system, it being possible for the solution to comprise other adjuvants, for example pearlescent agents.

In that embodiment, the coloured polymer is present in an amount of approximately from 0.1 to 5% by weight.

The cosmetic preparations and formulations according to the invention may also be used for colouring the hair, in which case they are used in the form of shampoos, creams or gels that are composed of the base substances conventionally employed in the cosmetics industry and a pigment according to the invention.

The cosmetic preparations and formulations according to the invention are prepared in conventional manner, for example by mixing or stirring the components together, optionally with heating so that the mixtures melt.

The Examples that follow illustrate the invention without limiting the scope thereof. Unless otherwise indicated, percentages and parts are percentages and parts by weight, respectively.

### EXAMPLES

### Example 1

A layer of about 50 nm of NaCl is vapor-deposited onto a metallic carrier In a vacuum chamber at a pressure of less than about 10⁻² Pa. At the same pressure, the following materials are then vapor-deposited in succession: SiO, Al and SiO, thereby producing on the metal belt a film having the following layer structure:

SiO (270 nm)/Al (40 nm)/SiO (270 nm).

The separating agent is then dissolved in water, whereupon flakes separate from the substrata. The resulting suspension is concentrated by filtration at atmospheric pressure and rinsed several times with deionised water in order to remove any Na⁺ and Cl⁻ ions present. After drying, SiO-coated aluminum flakes displaying lustrous metallized colors and having a brighter appearance and a greater brilliance in comparison with aluminum flakes known from the prior art are obtained.

To increase fastness to weathering and light, the pigments, in the form of loose material, may be heated at 200° C. for 2 hours in an oven through which air heated to 200° C. is passed.

1. An aluminum flake comprising
(A1) a layer consisting of SiO_{z},
(B) a layer consisting of aluminum on the layer (A1) and
(A2) a layer consisting of SiO_{z} on the layer (B), wherein 0.70≦z≦2.0.

2. An aluminum flake according to claim 1, comprising
(C1) a layer consisting of SiO₂,
(A1) a layer consisting of SiO_{y} on the layer (C1),
(B) a layer consisting of aluminum on the layer (A1),
(A2) a layer consisting of SiO_{y} on the layer (B) and
(C2) a layer consisting of SiO₂ on the layer (A2), wherein 0.95≦y≦2.0.

3. An aluminum flake comprising
(D1) a layer consisting of SiO₂,
(B) a layer consisting of aluminum on the layer (D1) and
(D2) a layer consisting of SiO₂ on the layer (B), wherein the layer thickness of the SiO₂ layer is from 200 to 500 nm.

4. An aluminum flake according to claim 1, wherein the layer thickness of the layer (B) consisting of aluminum is from 10 to 100 nm.

5. An aluminum flake according to claim 1, wherein the layer thickness of the layers (A1) and (A2) consisting of SiO_{z}, is from 200 to 350 nm.

6. A pigment based on the aluminum flakes according to claim 1, comprising on the layers (A1) and (A2) or over the entire surface of the aluminum flakes a layer (E) consisting of a dielectric material having a “high” refractive index.

7. A pigment based on the aluminum flakes according to claim 1, comprising over the entire surface of the aluminum flakes a layer (F) consisting of from 50 to 95% by weight carbon, from 5 to 25% by weight nitrogen and from 0 to 25% by weight of the elements hydrogen, oxygen and/or sulfur, the percentage by weight data relating to the total weight of the layer (F).

8. A pigment according to claim 6, wherein the layer thickness of the layer (E) is from 10 to 150 nm.

9. (canceled)

10. A paint, electrostatic coating, in ink-jet printing, cosmetic, coating, printing ink, plastics material, glaze for ceramics and glass, or security printing composition comprising an aluminum flake according to claim 1.

11. A paint, electrostatic coating, in ink-jet printing, cosmetic, coating, printing ink, plastics material, glaze for ceramics and glass, or security printing composition comprising a pigment according to claim 6.

12. An aluminum flake according to claim 3, wherein the layer thickness of the SiO₂ layers (D1) and (D2) is from 200 to 350 nm.

13. An aluminum flake according to claim 1, wherein the layer thickness of the layer (B) consisting of aluminum is from 30 to 50 nm.

14. An aluminum flake according to claim 3, wherein the layer thickness of the layer (B) consisting of aluminum is from 10 to 100 nm.

15. An aluminum flake according to claim 2, wherein the layer thickness of the layers (A1) and (A2) consisting of SiO_{y}, and the layer thickness of the layers (C1) and (C2) consisting of SiO₂ is from 200 to 350 nm.

16. A pigment based on the aluminum flakes according to claim 6, wherein the layer (E) consists of TiO₂ or carbon.

17. A pigment based on the aluminum flakes according to claim 2, comprising on the layers (A1) and (A2) or on the layers (C1) and (C2) or on the layers (D1) and (D2) or over the entire surface of the aluminum flakes a layer (E) consisting of a dielectric material having a “high” refractive index.

18. A pigment based on the aluminum flakes according to claim 2, comprising over the entire surface of the aluminum flakes a layer (F) consisting of from 50 to 95% by weight carbon, from 5 to 25% by weight nitrogen and from 0 to 25% by weight of the elements hydrogen, oxygen and/or sulfur, the percentage by weight data relating to the total weight of the layer (F).

19. A pigment according to claim 6, wherein the layer thickness of the layer (E) is from 30 to 70 nm.

20. A pigment according to claim 7, wherein the layer thickness of the layer (F) is from 10 to 150 nm.

21. A pigment according to claim 7, wherein the layer thickness of the layer (F) is from from 30 to 70 nm.
